SEQUENCE LISTING (2 sequences)

SEQ ID NO: 1 - DNA, 20 bp, Artificial Sequence
  Description of Artificial Sequence Oligonucleotide to act as a primer for PCR
gagctgtccctctatcctcg

SEQ ID NO: 2 - DNA, 20 bp, Artificial Sequence
  Description of Artificial Sequence Oligonucleotide to act as a primer for PCR
tgacctcagctgtctgctcc

(19) United States
(12) Patent Application Publication
(10) Pub. No.: US 2004/0053345 A1
(43) Pub. Date: Mar. 18, 2004

(54) Marker for probing the therapeutic efficacy of drugs

(76) Inventors: Akira Yamauchi, Kagawa (JP); Koji Murao, Kagawa (JP); Nozomu Nishi, Kagawa (JP); Mitsuomi Hirashima, Kagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/383,073

(22) Filed: Mar. 7, 2003

(30) Foreign Application Priority Data
Sep. 9, 2002 (JP) 2002-263093

(51) Int. Cl. G01N 33/574; C07K 14/47

(52) U.S. Cl. 435/7.23; 530/350

(57) ABSTRACT

Although adjuvant, or additional, therapy after surgery (e.g., adjuvant, or additional, hormonal therapy after breast cancer surgery) is performed to prevent postoperative recurrence of malignant tumors such as breast cancer, the incidence of disease recurrence still remains even if a representative drug, tamoxifen, commonly used is given. Thus, if it is possible to predict whether malignant tumors are reliably curable with tamoxifen or whether therapeutic drugs other than tamoxifen should be selected or not against such malignant tumors, it will greatly assist in the therapeutic guideline for preventing postoperative recurrence. There is a possibility that menin regulates the ER transcription activity in breast cancer cells. The expression of menin is remarkably responsible for the efficacy of estrogen antagonists (e.g., tamoxifen) having an inhibitory activity on estrogen binding to the ER. Techniques utilizing the present findings are provided. That is, provided is use of menin and menin expression gene as markers for probing the efficacy of therapeutic drugs (e.g., anti-cancer agents, antineoplastic agents) on tumor cells (e.g., breast cancer) or drug resistance markers for such therapeutic drugs, related reagents and methods for measurement/detection as well as systems utilizing them.

### FIELD OF THE INVENTION

The present invention relates to markers allowing us to evaluate whether or not a drug used for therapy can be effective in the treatment of tumor cells, for example, breast cancer, and to evaluate the sensitivity degree of tumor cells against the therapeutic drug as well as to methods for evaluating or assessing an efficacy of the therapeutic drug and systems utilizable therefor.

The present invention also relates to the use of menin and menin expression gene as markers for predicting or probing the efficacy of a therapeutic drug including, for example, an anti-cancer drug and an antineoplastic agent, in tumor cells such as breast cancer and as markers for drug-resistance with regard to cancer cells. The present invention relates to assay reagents for measurement/detection related thereto, measuring/detecting (or assaying) methods and assay systems utilized therefor.

### BACKGROUND OF THE INVENTION

The greatest factor in preventing postoperative recurrence in malignant tumors, particularly breast cancers is not surgical operation but postoperative adjuvant therapy (breast cancer postoperative adjuvant hormone therapy). Nearly all patients with breast cancer undergo adjuvant therapies after operation. The current adjuvant therapies are primarily hormone therapy, chemical therapy and a combination of both. Among them, the postoperative hormone therapy is effective only for estrogen (female hormone) receptor (ER) positive cases where estrogen dependent growth is shown. The positive rate of ER is about 60% in breast cancer.

In such a therapy, when tamoxifen which is a representative drug is administered to patients with ER-positive for 5 years, the recurrence rate declines to 50% of that in non-administered patients, but does not reduce to 0%. That is, even if patients take tamoxifen, there are some patients who have recurrence. Therefore, it appears that if the efficacy of tamoxifen can be predicted before tamoxifen is taken, unprofitable 5 years need not to be spent. However, at this moment, there is no report of a tamoxifen resistant factor, i.e., a predicting factor of tamoxifen efficacy in ER-positive patients.

Menin is known to be a gene product of a responsible gene, MEN 1, for multiple endocrine neoplasia type I (men 1) (pituitary tumors+pancreatic islet tumors+parathyroid hyperplasia), to exist in the nucleus of adenoma cells, and to suppress transcription activity of Jun D and the like (that is, to be considered as α-tumor suppressor gene). Menin is highly involved in tumorgenesis of endocrine glands. In other words, it has been thought that gene aberrance of menin may cause tumorgenesis of endocrine cells.

Unknown is how menin acts in malignant tumors, particularly breast cancer, and the relationship of menin to the drugs used for postoperative adjuvant therapy. On the other hand, since menin exists in the nucleus, if the relationship of menin to malignant tumors, particularly breast cancer, is demonstrated and if problems in the above postoperative adjuvant therapy are resolved, menin has a possibility to become highly helpful in establishing a definite therapeutic guideline in the prevention of the postoperative recurrence of malignant tumors, particularly breast cancer.

### SUMMARY OF THE INVENTION

The present inventors have carried out an extensive research for the purpose of discovering how menin (a gene product of men1, a responsible gene for multiple endocrine neoplasia type I), exists in nuclei of adenoma cells, and suppresses the transcription activity of Jun D and the like, acts in malignant tumors, particularly breast cancer. Especially, since menin exists in nuclei, a functional .association of menin with breast cancer cells was examined. In other words, since menin exists in nuclei, on the assumption that, if menin is expressed in nuclei of breast cancer cells, menin would potentially control the transcription function for an intranuclear receptor, ER, the study was carried forward. As a result, the inventors have found that there is a possibility that menin regulates an ER transcription activity in breast cancer cells, and that menin is highly involved in the effects or actions of estrogen antagonists, e.g., tamoxifen (Nolvadex), having an activity to inhibit the binding of estrogen to the ER, and have succeeded in providing techniques utilizing these findings.

The present invention provides:

(1) use of menin as a marker for probing or assessing the therapeutic efficacy of antineoplastic drugs for malignant tumor cells including breast cancer;

(2) the use of menin according to the above (1), wherein the antineoplastic drugs are hormonal drugs used for adjuvant, or additional, therapy after surgery or radiotherapy (e.g., adjuvant, or additional, hormonal therapy after breast cancer surgery) to prevent malignant tumor (particularly breast cancer) recurrence after surgery or radiotherapy;

(3) the use of menin according to the above (1) or (2), wherein the drug is selected from the group consisting of estrogen antagonists, LH-RH agonists and aromatase inhibitors;

(4) the use of menin according to any of the above (1) to (3), wherein the drug is selected from the group consisting of active agents capable of binding to estrogen receptor (ER) so as to inhibit an estrogen binding to the ER, suppressors against estrogen synthesis in the ovary and inhibitory agents against estrogen synthesis in fat tissues;

(5) the use of menin according to any of the above (1) to (4), wherein the drug is selected from the group consisting of tamoxifen, toremifene, goserelin, leuprorelin, anastrozole, letrozole and exemestan;

(6) a method for selecting or determining a therapeutic drug or predicting or assessing the therapeutic efficacy of said drug, which comprises using menin as a marker for probing or examining the therapeutic efficacy upon the selection of therapeutically-effective drugs for the adjuvant, or additional, hormonal therapy after breast cancer surgery;

(7) a method for probing and/or predicting the therapeutic efficacy of a therapeutic drug, which comprises testing or examining the presence or absence of menin (including the presence or absence of menin gene expression) in breast cancer cells and using menin as a marker so as to predict or forecast the efficacy of an estrogen antagonist (such as tamoxifen) on the hormonal therapy;

(8) the method according to the above (7), wherein the test or examination is a member selected from the group consisting of immunohistological staining, immunohistochemistry using immunoenzyme techniques, enzyme immunoassays, immunoblotting (Western blot) and immunoprecipitation wherein anti-menin antibody is used; and Northern blotting, RT-PCR and in situ hybridization wherein cDNA is used;

(9) a reagent for probing or assessing a therapeutically-effective drug on adjuvant, or additional, hormonal therapy after breast cancer surgery, which comprises an element employing menin as a marker for probing or examining the therapeutic efficacy of said drug, said reagent being used for measuring or assaying menin in a subject sample to probe or determine the therapeutic validity of said drug on adjuvant, or additional, hormone therapy after breast cancer surgery;

(10) the reagent according to the above (9), wherein primers for the amplification of menin gene or probes for the menin gene are contained;

(11) the reagent according to the above (9), wherein an anti-menin antibody is contained; and

(12) a method for selecting a therapeutic drug for breast cancer, which comprises, when breast cancer cells are menin positive in their nuclei, avoiding the use of tamoxifen and other therapeutic drugs which block the ER and administering an LH-RH analog (for premenopausal patients) or an aromatase inhibitor (for postmenopausal patients), which inhibits estrogen synthesis per se.

The above objectives and other objectives, features, advantages, and aspects of the present invention are readily apparent to those skilled in the art from the following disclosures. It should be understood, however, that the description of the specification including the following best modes for carrying out the invention, examples, etc. is illustrating preferred embodiments of the present invention and given only for explanation thereof. It will become apparent to the skilled in the art that a great number of variations and/or alterations (or modifications) of this invention may be made based on knowledge from the disclosure in the following parts and other parts of the specification without departing from the spirit and scope thereof as disclosed herein. All of the patent publications and reference documents cited herein for illustrative purposes are hereby incorporated by reference into the present disclosure.

### BRIEF DESCRIPTION OF THE DRAWINGS

FIG. 1 shows the results of examining the expression of menin in breast cancer cells by RT-PCR and Western blotting.

FIG. 2 shows the results of examining the regulation of menin on ER transcription activity by the reporter gene assay with ERE-luc.

FIG. 3 shows the results of examining the regulation of menin on ER transcription activity in the presence of tamoxifen by the reporter gene assay with ERE-luc.

FIG. 4 shows the results of GST-pull down assay.

FIG. 5 shows the results of immunohistological staining with anti-menin antibodies.

FIG. 6 shows the relationship between menin expression and various factors.

FIG. 7 shows the results obtained by assessment for menin-biding domains of the ER.

### DESCRIPTION OF THE PREFERRED EMBODIMENTS

Focusing attention on the relationship between menin and the estrogen receptor (ER), examined was its functional relation to the ER on tumor cells, particularly breast cancer cells. How menin acts on the ER transcription function can be examined, for example, by introducing wild-type and variant-type menin genes into the breast cancer cell line, MCF-7, and simultaneously introducing, as a reporter gene, ERE-luc (estrogen receptor binding element labeled with fluorescent luciferase), followed by conducting a reporter gene assay in a system where, as the transcription progresses, a fluorescent intensity is increased.

The wild type menin promotes the ER transcription function while the variant type does not. When an ER gene is introduced into monkey cells, COS-7 cells, in addition to the above genes to examine whether the transcription is promoted by three introduced genes alone or not, the wild type menin promotes the ER transcription function but the variant type does not as is the case with MCF-7 cells. Moreover, when tamoxifen was added to the MCF-7 system, no suppressive effect of tamoxifen on the transcription was observed. Therefore, it is apparent that menin can be a tamoxifen resistant factor. To carry out GST-pull down assay in order to examine a physical binding of the ER with menin, when recombinant wild type menin and variant menin are reacted with an ER-immobilized column, the variant menin does not bind to the ER while the wild menin does bind. That is, it has been found that menin promotes the transcription via binding to the ER.

In the present invention, utilizing “gene recombination techniques”, not only menin peptides and fragments thereof, and nucleic acids encoding the same can be obtained, isolated, sequenced, and identified but also recombinants thereof can be constructed and produced. Gene recombination techniques (including recombinant DNA techniques) as can be used herein include those known in the art, and can be carried out by the methods described in, for example, Sambrook, J., Fritsch, E. F. & Maniatis, T., “Molecular Cloning: A Laboratory Manual (2nd edition)”, Cold Spring Harbor Laboratory Press, Cold Spring Harbor, N.Y., 1989; Glover, D. M. et al. ed., “DNA Cloning”, 2nd ed., Vol. 1 to 4 (The Practical Approach Series), IRL Press, Oxford University Press, 1995; “Zoku-Seikagaku Jikken Koza 1, Idenshi Kenkyuhou II” ed., by Japanese Biochemical Society, Tokyo Kagaku-dojin Publishing Co., Inc., 1986; “Shin-Seikagaku Jikken Koza 2, Kakusan III (Recombinant DNA technique)” ed., by Japanese Biochemical Society, Tokyo Kagaku-dojin Publishing Co., Inc., 1992; Wu, R., ed., “Methods in Enzymology”, Vol. 68 (Recombinant DNA), Academic Press, New York, 1980; Wu, R. et al., ed., “Methods in Enzymology”, Vol. 100 (Recombinant DNA, Part B) & 101 (Recombinant DNA, Part C), Academic Press, New York, 1983; Wu, R. et al., ed., “Methods in Enzymology”, Vol. 153 (Recombinant DNA, Part D), 154 (Recombinant DNA, Part E) & 155 (Recombinant DNA, Part F), Academic Press, New York, 1987; Miller, J. H., ed., “Methods in Enzymology”, Vol. 204, Academic Press, New York, 1991; Wu, R. et al., ed., “Methods in Enzymology”, Vol. 218, Academic Press, New York, 1993; Weissman, S., ed., “Methods in Enzymology”, Vol. 303, Academic Press, New York, 1999; Clorioso, J. C., ed., “Methods in Enzymology”, Vol. 306, Academic Press, New York, 1999 etc., or by methods described in the references cited therein or methods substantially equivalent thereto or modified methods thereof (the disclosures of which are incorporated herein by reference).

The term “polymerase chain reaction” or “PCR” used herein usually refers to techniques described in U.S. Pat. No. 4, 682,195. For example, the PCR is an in vitro method for the enzymatic amplification of desired specific nucleotide sequences. In general, the PCR includes repetitive series of cycles wherein a primer elongation synthesis is constructed using two oligonucleotide primers capable of preferentially hybridizing with a template nucleic acid. Typically, the primers used in PCR may include those which are complementary to the internal nucleotide sequence of interest in the template. For example, preferable primer pairs as used herein may be those which are complementary to both ends of said nucleotide sequence to be amplified, or flanking regions adjacent to said nucleotide sequence. It is preferred to select a 5′-terminal primer such that at least an initiation codon is contained or the amplification can be performed including the initiation codon, and to select a 3′-terminal primer such that at least a stop codon is contained or the amplification can be performed including the stop codon. The primers include oligonucleotides made up of preferably 5 or more bases, more preferably 10 or more bases, and still preferably 18 to 25 bases.

The PCR reaction can be carried out by methods known in the art or methods substantially equivalent thereto and modified methods thereof, and can be performed according to methods described, for example, in R. Saiki et al., Science, 230: 1350, 1985; R. Saiki et al., Science, 239: 487, 1988; H. A. Erlich (ed.), PCR Technology, Stockton Press, 1989; D. M. Glover et al. (ed.), “DNA Cloning”, 2nd ed., Vol. 1, (The Practical Approach Series), IRL Press, Oxford University Press (1995); M. A. Innis et al. (ed.), “PCR Protocols: A Guide to Methods and Applications”, Academic Press, New York (1990)); M. J. McPherson, P. Quirke and G. R. Taylor (ed.), PCR: A Practical Approach, IRL Press, Oxford (1991); M. A. Frohman et al., Proc. Natl. Acad. Sci. USA, 85, 8998-9002 (1988) and the like or modified or altered methods thereof. The PCR methods can also be performed using commercially available kits suitable therefor, and can also be carried out according to protocols demonstrated by kit manufacturers or kit distributors.

For the PCR reaction, in a representative case, for example, a template (e.g., DNA synthesized using mRNA as a template; 1st strand DNA) and primers synthesized according to designs on said gene are mixed with a 10× reaction buffer (attached with a Taq DNA polymerase kit), dNTPs (deoxyribonucleoside triphosphates; dATP, dGTP, dCTP and dTTP mix), Taq DNA polymerase and deionized distilled water. The mixture is subjected to 25 to 60 cycles of amplification using an automated thermal cycler such as GeneAmp 2400 PCR system, Perkin-Elmer/Cetus under general PCR cycle conditions. The number of amplification cycles can be suitably set to an appropriate value depending on purposes. The PCR cycle includes, for example, denaturation at 90 to 95° C. for 5 to 100 sec, annealing at 40 to 60° C. for 5 to 150 sec and extension at 65 to 75° C. for 30 to 300 sec, and preferably denaturation at 94° C. for 15 sec, annealing at 58° C. for 15 sec and extension at 72° C. for 45 sec. For the annealing temperature and reaction time, an appropriate value is suitably selected by experimentation. For the denaturation and extension time, an appropriate value suitably varies according to the strand length of expected PCR products. In general, the time of annealing preferably varies depending on the Tm value of primer-template DNA hybrids. The time period of extension is usually set with the aim of getting about 1 min per 1000 bp in strand length, but it may be possible to select a shorter time period in some cases.

The term “oligonucleotide(s)” used herein refers to a relatively short single-stranded polynucleotide or double-stranded polynucleotides, or preferably polydeoxynucleotide(s). They can be chemically synthesized by known methods as described in Angew. Chem. Int. ed. Engl., Vol. 28, p716-734 (1989), including the phosphotriester method, the phosphodiester method, the phosphite method, the phosphoamidite method, the phosphonate method and the like. It is known that the synthesis can be conveniently carried out on modified solid supports. For example, the synthesis can be carried out using an automated synthesizer and such a synthesizer is commercially available. The oligonucleotide may contain one or more modified nucleotide bases. For example, it may contain a nucleotide base which does not naturally occur, such as inosine, or a tritylated nucleotide base, etc. In some cases, it may contain a marker-tagged nucleotide base.

Herein, to analyze expressed proteins, polypeptides with mutations can be prepared according to techniques usually applied with the standard gene engineering methods based on the nucleotide sequence of the given gene. They are mutated polypeptides wherein one or more amino acid residues are appropriately substituted, deleted, inserted, translocated, rearranged or added with regard to the amino acid sequence of the given polypeptide and whose sequence is homologous to the original sequence. Such mutations, conversions and modifications include the methods described in, for example, “Zoku-Seikagaku Jikken Koza 1, Idenshi Kenkyuhou II” Japanese Biochemical Society ed., p.105 (Susumu Hirose), Tokyo Kagaku-dojin Publishing Co., Inc. (1986); “Shin-Seikagaku Jikken Koza 2, Kakusan III (Recombinant DNA technique)” Japanese Biochemical Society ed., p.233 (Susumu Hirose), Tokyo Kagaku-dojin Publishing Co., Inc. (1992); “Methods in Enzymology” Vol. 154, pp.350 & 367, R. Wu, L. Grossman ed., Academic Press, New York (1987); “Methods in Enzymology” Vol. 100, pp.457 & 468, R. Wu, L. Grossman ed., Academic Press, New York (1983); J. A. Wells, et al., Gene, 34:315, 1985; T. Grundstroem et al., Nucleic Acids Res., 13:3305, 1985; J. Taylor et al., Nucleic Acids Res., 13:8765, 1985; “Methods in Enzymology” Vol. 155, p.568, R. Wu ed., Academic Press, New York (1987); A. R. Oliphant et al., Gene, 44:177, 1986, etc. Included are, for example, methods such as the site-directed mutagenesis (site specific mutagenesis) utilizing synthetic oligonucleotides (Zoller et al., Nucl. Acids Res., 10:6487, 1987; Carter et al., Nucl. Acids Res., 13:4331, 1986), cassette mutagenesis (Wells et al., Gene, 34:315, 1985), restriction selection mutagenesis (Wells et al., Philos. Trans. R. Soc. London Ser. A, 317:415, 1986), alanine-scanning (Cunningham & Wells, Science 244:1081-1085, 1989), PCR mutagenesis, Kunkel method, dNTP[a S] method (Eckstein), area specific mutagenesis using sulfurous or nitrous acid, and the like.

The polypeptides (proteins) may be expressed as fusion polypeptides (fusion proteins) when made by gene recombination methods, and may be converted or processed into those having in vivo or in vitro substantially equivalent biological activity as compared to the desired polypeptide. The fusion production usually used in gene engineering can be used. Such fusion polypeptides can be purified by an affinity chromatography taking advantage of their fusion moieties. Such fusion polypeptides include those fused to a histidine tag, or those fused to the amino acid sequence of β-galactosidase (β-gal), maltose-binding protein (MBP), glutathione S-transferase (GST), thioredoxin (TRX) or Cre recombinase. Similarly, the polypeptide can be provided with a heterogenous epitope tag, and can be isolated/purified by an immunoaffinity chromatography using an antibody specifically binding to the epitope. In the more suitable embodiments, the representatives include a poly-histidine (poly-His) or poly-histidine-glycine (poly-His-Gly) tag. The epitope tag includes, for example, AU5, c-Myc, CruzTag 09, CruzTag 22, CruzTag 41, Glu-Glu, HA, Ha.11, KT3, FLAG (registered trademark, Sigma-Aldrich), Omni-probe, S-probe, T7, Lex A, V5, VP16, GAL4, VSV-G and the like (Field et al., Molecular and Cellular Biology 8: pp.2159-2165, 1988; Evans et al., Molecular and Cellular Biology 5: pp.3610-3616, 1985; Paborsky at al., Protein Engineering 3(6): pp.547-553, 1990; Hopp et al., BioTechnology 6: pp.1204-1210, 1988; Martin et al., Science 255:192-194, 1992; Skinner et al., J. Biol. Chem., 266: pp.15163-15166, 1991; Lutz-Freyermuth et al., Proc. Natl. Acad. Sci. USA, 87: pp.6393-6397, 1990). Two-hybrid method utilizing yeast can also be applied.

Moreover, the fusion polypeptides can be those tagged with a marker such that they become detectable proteins. In more suitable embodiments, the detectable markers may be Biotin-Avi Tag which is a biotin/streptoavidin system and fluorescent substances. The fluorescent substances include green fluorescent proteins (GFP) derived from luminescent jelly fish such as Aequorea victorea and the like, modified variants thereof (GFP variants) such as EGFP (enhanced-humanized GFP) and rsGFP (red-shift GFP), yellow fluorescent proteins (YFP), green fluorescent proteins (GFP), cyan fluorescent proteins (CFP), blue fluorescent proteins (BFP), GFP derived from Renilla reniformis, and the like (Atsushi Miyawaki ed., Jikken Igaku, suppl., Postgenome Jidai no Jikken Kouza 3. GFP and Bioimaging, Youdosha, 2000). Also, detection can be carried out using antibodies (including monoclonal antibodies and fragments thereof) which specifically recognize the above fusion tag. Expression and purification of such fusion polypeptides can be carried out using commercially available kits suitable therefor, and can also be conducted according to protocols demonstrated by kit manufacturers or kit dealers.

The resultant proteins (may include peptides and polypeptides) can be immobilized in combination with an appropriate carrier or solid phase by the known techniques such as enzyme immunoassays. Immobilized proteins and peptides can be conveniently utilized for binding assays and screening for substances.

Modifications and alterations of the polypeptide and protein structures can be performed in reference to “Shin-Seikagaku Jikken Koza 1, Protein VII, Protein Engineering” Japanese Biochemical Society ed., (Tokyo Kagaku-dojin Publishing Co., Inc., 1993) using the methods described therein or the methods described in the references cited therein, and, further, the methods substantially equivalent thereto. The modification and alteration may be deamination, hydroxylation, carboxylation, phosphorylation, sulfation, alkylation such as methylation, acylation such as acetylation, esterification, amidation, ring-opening, cyclization, glycosylation, alteration of contained saccharide chains to different types, increasing or decreasing the number of contained saccharide chains, lipid bond, substitution to D-amino acid residues, etc. Those methods are known in the art (For example, T. E. Creighton, Proteins: Structure and Molecular Properties, pp.79-86, W. H. Freeman & Co, San Francisco, USA, 1983, etc.).

The present invention provides a detection agent for selecting and/or probing the therapeutic drug for tumor cells as well as a detection method for probing the efficacy of the therapeutic drug for tumor cells and a system utilizing the same characterized in that nucleic acids hybridizable with nucleic acids encoding menin or constitutive domains thereof are contained as an active ingredient. PCR methods, and further PCR methods using reverse transcriptase (RT-PCR) can be utilized for isolation of the gene. The hybridizing nucleic acids include, for example, probes, primers and the like. Nucleic acids can be utilized without any limitation if they are probes hybridizing to the menin gene or the products thereof so far as they meet the purpose. The nucleic acids are available according to “gene recombination techniques” described above, and can be easily obtained by, for example, utilizing the information for the base sequence of menin known in the art, designing and synthesizing multiple primers and performing PCR (polymerase chain reaction). Manufacturing the primers can be performed by the methods known in the art, can be synthesized representatively by the phosphodiester method, phosphotriester method, phosphoamidite method and the like, and can be synthesized, for example, by an automatic DNA synthesizer, e.g., model 381A DNA synthesizer (Applied Biosystems). PCR can be carried out using a cDNA library, a sense primer and an antisense primer to amplify a cDNA. The resultant nucleic acids can be utilized as specific hybridization probes. To label the probe with radioisotopes and so on, the labeling can be carried out using commercially available kits, e.g., random prime DNA labeling kit (Boehringer Mannheim). For example, the probe having radioactivity can be obtained by labeling DNA for the probe with [α-³²P]dCTP (Amersham) using a random prime kit (Pharmacia LKB, Uppsala). Also, those known in the art can be used as labels for the probes, and those can be used by appropriately selecting from the labels described in the section of the antibodies.

Hybridization is carried out by transferring a sample retaining the given DNA on a membrane such as a nylon filter, which is subjected to a denature treatment, fixation treatment, washing treatment and the like if necessary, and subsequently reacting with a labeled probe DNA fragment denatured if necessary in a buffer for the hybridization. The hybridization treatment is carried out usually at approx. 35 to 80° C., more suitably at approx. 50 to 65° C. for approx. 15 min to 36 hours, more suitably approx. 1 hour to 24 hours, but can be carried out by selecting an appropriately optimal condition. For example, the hybridization is carried out at approx. 55° C. for approx. 18 hours. The buffer for the hybridization can use those selected from those usually used in the art. For example, a rapid hybridization buffer (Amersham) and the like can be used. Denature treatment of the transferred membrane includes methods using an alkali denature solution, and it is preferable to treat with a neutralization solution or buffer following that treatment. Fixation treatment of the membrane is carried out by baking usually at approx. 40 to 100° C., more suitably at approx. 70 to 90° C. for approx. 15 min to 24 hours, more suitably for approx. 1 hour to 4 hours, but can be carried out by appropriately selecting a preferable condition. For example, the filter is fixed by baking at approx. 80° C. for approx. 2 hours. Washing treatment of the transferred membrane can be carried out by washing with a washing solution usually used in the art such as Tris-HCl buffer, pH 8.0 containing 1M NaCl, 1 mM EDTA and 0.1% sodium dodecyl sulfate (SDS), and the like. As membranes such as nylon filters those selected from those usually used can be included, and for example, nylon filter [Hybond-N] (Amersham) and the like can be included.

The above alkali denature solution, neutralization solution and buffer can be used by selecting those usually used in the art, the alkali denature solutions include, for example, a solution containing 0.5M NaOH and 1.5M NaCl and the like, the neutralization solution can include, for example, 0.5M Tris-HCl buffer (pH 8.0) containing 1.5M NaCl and the like, and the buffer can include, for example, 2×SSPE (0.36M NaCl, 20 mM NaH₂PO₄ and 2 mM EDTA) and the like. It is preferred that prehybridization treatment of the transferred membrane is performed for the transferred membrane prior to the hybridization treatment, if necessary, to prevent non-specific hybridization reactions. The prehybridization treatment can be carried out, for example, by immersing in the prehybridization solution [50% formamide, 5× Denhardt's solution (0.2% bovine serum albumin, 0.2% polyvinyl pyrrolidone), 5×SSPE, 0.1% SDS, 100 μg/ml heat-denatured salmon sperm DNA] and reacting at approx. 35 to 50° C., preferably at approx. 42° C. for approx. 4 to 24 hours, preferably approx. 6 to 8 hours, but those skilled in the art can determine more preferable conditions by appropriately repeating experiments for these conditions. The denature of the labeled probe DNA fragments used for the hybridization can be carried out by, for example, heating at approx. 70 to 100° C., preferably at 100° C. for approx. 1 min to 60 min, preferably for approx. 5 min. The hybridization can be carried out by methods known in the art or methods following thereto. Herein, a stringent condition indicates, for example, the condition where the concentration of sodium is approx. 15 to 50 mM, preferably approx. 19 to 40 mM, more preferably approx. 19 to 20 mM, and the temperature is approx. 35 to 85° C., preferably approx. 50 to 70° C., more preferably approx. 60 to 65° C.

After completion of the hybridization, the filter is thoroughly washed to remove labeled probes other than the labeled probe DNA fragments having the specific hybridization reaction completed. The washing of the filter can be carried out by selecting among those usually used in the art and, for example, can be performed by washing with a 0.5×SSC solution (0.15M NaCl, 15 mM citric acid) containing 0.1% SDS.

Hybridized portions can be detected representatively by autoradiography, but those appropriately selected among the methods used in the art can also be used for detection.

Validity of anti-tumor agents, anti-cancer agents and adjuvant hormone therapeutic drugs for breast cancer and the like can be detected by detecting/determining the menin expressing genes (including DNA such as cDNA and RNA such as mRNA) according to “gene recombination techniques” described above, by the techniques known for detecting/determining the expression of the certain gene in the art, such as in situ hybridization, Northern blotting, dot blotting, RNase protection assay, RT-PCR, real-time PCR (Journal of Molecular Endocrinology 25:169-193, 2000, and the references cited therein), DNA array analysis (Mark Shena ed., “Microarray Biochip technology”, Eaton Publishing, March, 2000), and the like. A menin expressing gene determination system utilizing such technologies, reagents, methods and processes utilized therefor all are included in detection agents for probing the selection of the therapeutic drugs for tumor cells, detection methods for probing the efficacy of the therapeutic drugs for tumor cells, and the system utilized therefor of the present invention. The in situ hybridization may include, for example, non-R1 in situ hybridization, and may include, for example, direct and indirect methods. The direct method uses those where a detectable molecule (reporter) is directly bound to a nucleic acid probe, whereas the indirect method is, for example, where a signal is amplified using an antibody against a reporter molecule.

Functional groups (e.g., primary aliphatic amino group, SH group, etc.) are introduced into the oligonucleotide in the nucleic acid probe, and hapten, fluorescent dye, enzymes and the like may be bound to such functional groups. Labels of the nucleic acid probe include representatively digoxigenin (DIG), biotin, fluorescein and the like, those appropriately selected from the labels described in the section of antibodies mentioned above can be used, and multiple labeling can also be utilized, and further labeled antibodies can also be utilized. Those appropriately selected among the methods known in the art can be used as the labeling methods for the nucleic acid probes, and include, for example, random prime method, nick translation method, DNA amplification by PCR, labeling/tailing method, in vitro transcription method and the like. For observation of the treated samples, those appropriately selected among the methods known in the art can be used, and for example, a dark field microscopy, a phase contrast microscopy, a reflection contrast microscopy, a fluorescent microscopy, a digital imaging microscopy, an electron microscopy and the like can be used, and furthermore the observation can be performed by a flow cytometry.

In the present invention, menin and the menin expressing gene can be used as a marker having a property to resist anti-tumor agents or a marker for drug resistant tumors, thereby being capable-of making detection agents for therapy resistant cancers or detection and/or measuring agents for therapy resistant tumors, particularly detection methods for the efficacy of the adjuvant hormone therapeutic drugs in breast cancer or detection and/or measuring methods for the adjuvant hormone therapeutic drug resistant tumors, and sets or systems of reagents for detection of such cancers or detection and/or measurement of such tumors, resulting in not only serving as a definite diagnosis, prevention and therapy of problematic cancers but also being excellent. Moreover, after the treatment of cancer, i.e., for prognosis, they are capable of being made into detection agents of cancer features or detection and/or measuring agents for drug resistance of tumors, assay and/or measuring methods contributing to drug selection effective for cancers, and reagent sets or systems therefor, whereby prognostic excellent functions and action efficacy can be anticipated.

The term “marker” as used herein may indicate those capable of recognizing or identifying “an ability capable of evaluating an activity expression for the estrogen receptor” or “features of tumors in respect to ER”, further may be those indicating the degree of sensitivity for adjuvant hormone therapeutic drugs of the “tumor” and the action as a measure of malignancy of “tumors” for adjuvant hormone therapeutic drugs, and may be considered to be referred to those exhibiting the above functions due to the presence or absence of and/or the quantitative difference of the markers.

For menin and the related proteins, the fragments thereof, and further the nucleic acids including DNA (also including mRNA and oligonucleotides) related thereto, their expressions can be identified and assessed using them alone or organically, further in appropriate combination with an antisense method, antibodies including monoclonal antibodies and technologies for transgenic animals, and by being applied for genomics and proteomics technologies. For example, according to the present invention, the menin variants are available for functional analyses utilizing a dominant negative effect. Also, they are applied for RNAi (RNA interference) technology using double strand RNA (dsRNA). Thus, gene polymorphism analyses mainly including single nucleotide polymorphism (SNP), gene expression analyses using nucleic acid arrays and protein arrays, gene function analyses, protein-protein interaction analyses, related disease analyses and disease therapeutic drug analyses are capable of being carried out. For example, in the nucleic acid array technology, samples are analyzed using cDNA libraries, arranging DNA obtained by PCR on the substrate at a high density using a spotting apparatus, and utilizing hybridization.

The arraying can be performed by adhering DNA at each inherent site on the substrate such as slide glass, silicon plate, plastic plate and the like using needles or pins or with an ink jet printing technology. Data is acquired by observing signals obtained from the results of hybridization on the nucleic acid arrays. The signals may be those obtained from labels such as fluorescent dyes (e.g., Cy3, Cy5, BODIPY, FITC, Alexa Fluor dyes (brand name), Texas red (brand name). Laser scanner and the like can be used for the detection, and the obtained data can be processed by a computer system installed with programs according to an appropriate algorithm. Also in protein array technology, tagged recombinant expression protein products may be utilized, and two dimensional electrophoresis (2-DE), mass analyses (MS)(including technologies such as electrospray ionization method (ESI), matrix-assisted laser desorption/ionization method (MALDI), MALDI-TOF analyzer, ESI-triple quadruple mass spectrometer, ESI-ion trap mass spectrometer and the like) including enzyme digestion fragments, staining techniques, isotope labeling and analysis, image processing and the like can be utilized. Therefore, software and a database related to menin and antibodies thereto may be included in the present invention.

Hormone therapeutic drugs used for the postoperative adjuvant therapy (breast cancer postoperative adjuvant hormone therapy) for the prevention of the postoperative recurrence of malignant tumors, particularly breast cancer include, for example, estrogen antagonists, LH-RH agonist, aromatase inhibitors and the like. Such drugs include those having an activity inhibiting the binding of estrogen to estrogen receptor by binding to the estrogen receptor, those having an activity suppressing estrogen synthesis in the ovary, those having an inhibitory activity of estrogen synthesis in fat tissue, and the like. Preferably, those exhibiting effects such as suppressing growth of malignant tumor cells, particularly breast cancer cells are included. The hormone therapeutic drugs include, for example, tamoxifen (brand name: Nolvadex), toremifene (brand name: Fareston) which are known as estrogen antagonists, goserelin (brand name: Zoladex), leuprorelin (brand name: Leuplin) which are known as LH-RH agonists, and anastrozole (brand name: Arimidex), letrozole (brand name: Femara), exemestan (brand name: Aromathyn) which are known as aromatase inhibitors.

Thus, in the representative embodiment, according to the present invention, the presence or absence of menin (including the presence or absence of menin expression) in breast cancer cells can predict the efficacy of estrogen antagonists such as tamoxifen and the like on the hormone therapy. It can be considered that this is not limited to tamoxifen and that the efficacy prognostication of drugs (toremifene) having similar effects is possible. Therefore, the presence or absence of menin expression becomes an important criteria in decision making of drugs used for breast cancer adjuvant hormone therapy. Test methods for menin expression include the technologies known in the art, and include, for example, immunohistological staining methods, enzyme antibody methods (ELISA, etc.), immune blotting methods (western blotting method), immunoprecipitation methods which use anti-menin antibodies, Northern blotting method, RT-PCR methods, in situ hybridization which use cDNA, and the like. According to the present invention, in cases where menin is positive in the nuclei of breast cancer cells, an LH-RH analog (before menopause) or an aromatase inhibitor (after menopause) which inhibits estrogen synthesis per se is administered rather than tamoxifen which blocks the estrogen receptor.

Thus, in the case of a drug such as tamoxifen, since the improved effective rate of tamoxifen can be predicted, the use of tamoxifen can be reliably performed. In the case of the LH-RH analog or the aromatase inhibitor, since use is effective in place of tamoxifen which is conventionally widely used, effective therapy according to use can be developed. It has been elucidated that menin functions as a transcription factor of estrogen receptor residing in nuclei, and it has been demonstrated that menin is involved in the generation of various proteins associated with growth of breast cancer cells. Therefore, as the transcription of estrogen receptor advances by the action of menin, proliferative response is also facilitated. Thus, it is useful for menin to be used as a marker in the decision making of the drug used for breast cancer adjuvant therapy.

It can be said that because of binding to the estrogen receptor, menin is a resistant factor for those having an activity inhibiting the binding of estrogen to the estrogen receptor. All the technologies described above utilizing new findings are within the scope of the present invention.

In one aspect, the effectiveness of the invention includes those containing an anti-menin antibody as an active ingredient in the reagents and assays for probing the sensitivity of malignant tumors such as breast cancer against drugs, for example, a drug for hormone replacement therapy. Here, the anti-menin antibody can be obtained using means known in the art as a polyclonal or monoclonal antibody.

As used herein, the term “antibody” is used in the broadest sense and may cover a single species of desirable monoclonal antibodies against desired menin polypeptides and menin-related peptide fragments and antibody compositions having a specificity to various epitopes thereof, further monovalent or polyvalent antibodies and polyclonal and monoclonal antibodies, and also those which are intact molecules or fragments and derivatives thereof, including F(ab′)₂, Fab′ and Fab fragments, and also chimeric antibodies, hybrid antibodies each having at least two antigen or epitope binding sites, or bispecific recombinant antibodies (e.g., quadromes, triomes, etc.), interspecies hybrid antibodies, anti-idiotypic antibodies and those which have been chemically modified or treated and must be regarded as derivatives of these antibodies and further which may be produced either by adopting cell fusion or hybridoma techniques or antibody engineering or by using synthetical or semisynthetical techniques in known manner, which may be prepared either by the known conventional methods in view of antibody production or by recombinant DNA techniques, and which have neutralizing or binding properties with respect to the target antigen substances or target epitopes described and defined herein. Particularly preferable antibodies according to the present invention include those capable of specifically identifying and/or recognizing wild type menin polypeptides or polypeptides for the domains involved in the function as an ER transcription-coupling factor of the wild type menin.

In order to obtain anti-menin antibodies as polyclonal antibodies, mammalian or avian animals are immunized with menin, or a fragment thereof or a peptide of the partial menin sequence which is an immunogen, and antisera are collected from the mammalian or avian animals. Then, the polyclonal antibodies contained in the antisera can be used.

Mammalian animals immunized with the menin as a sensitized antigen are not particularly limited, and generally rodent animals such as a mouse, rat, hamster, rabbit, sheep, goat, cattle, horse, pig, dog, cat, primates such as monkeys, avian animals such as chickens and the like are used. Moreover, animals are preferably selected by considering compatibility with parent cells used for cell fusion in some cases.

According to the methods known in the art, the animal is immunized with the sensitized antigen. For example, as a general method, immunization is carried out by injecting the sensitized antigen into the animal intraperitoneally or subcutaneously. An appropriate carrier can also be used for immunization with the sensitized antigen. The antiserum containing polyclonal antibodies can be prepared from the blood collected from the animal after the immunized animal is bred for a given period. The resultant antiserum is confirmed to recognize menin specifically, and then provided for the purposes of the invention.

First, menin used as the sensitized antigen for acquiring antibodies can be obtained by expressing the menin gene/amino acid sequence known in the art. That is, the gene sequence encoding menin or a partial domain thereof, a partial protein or polypeptide fragment of menin, a peptide having a partial amino acid sequence corresponding to the amino acid sequence of menin is inserted into an expression vector system known in the art to transform appropriate host cells, and subsequently, the target menin protein or partial domain protein thereof, the partial protein or polypeptide fragment of menin, the peptide having the partial amino acid sequence corresponding to the amino acid sequence of menin is purified from the host cells or the culture supernatant thereof by the methods known in the art.

In the present invention, those obtained as monoclonal antibodies derived from the mammalian animal can also be used as anti-menin antibodies.

Monoclonal antibodies prepared against antigenic substances are produced by any method capable of providing production of antibody molecules by a series of cell lines in culture. The modifier “monoclonal” indicates the character of the antibody as being obtained from a substantially homogeneous population of antibodies, and is not to be construed as requiring production of the antibody by any particular method. The individual monoclonal antibodies are those containing a population of identical antibodies except for possible naturally occurring mutations that may be present in minor amounts. Monoclonal antibodies are highly specific, being directed against a single antigenic site. In contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The monoclonal antibodies included within the scope of the invention include hybrid and recombinant antibodies. They are obtainable by substituting a constant domain of an antibody for a variable domain (e.g., “humanized” antibodies), or a heavy chain for a light chain, by substituting a chain from one species with a chain from another species, or by fusing to heterogeneous proteins, regardless of species of origin or immunoglobulin class or subclass designation, so long as they exhibit the desired biological activity (for example, U.S. Pat. No. 4,816,567; Monoclonal Antibody Production Techniques and Applications, pp.79-97, Marcel Dekker Inc., New York, 1987, etc.) Methods suitable for producing monoclonal antibodies include, for example, the hybridoma method (Kohler G. and Milstein C., Nature, 256: pp.495-497, 1975; the human hybridoma methods (Kozbor et al., Immunology Today, 4: pp.72-79, 1983; Kozbor, J. Immunol., 133: pp.3001, 1984; Brodeur et al., Monoclonal Antibody Production Techniques and Applications, pp.51-63, Marcel Dekker, Inc., New York, 1987; the trioma method; the EBV-hybridoma method (Cole et al., Monoclonal Antibodies and Cancer Therapy, Alan R. Liss, Inc., pp.77-96, 1985 (The Method for Producing a Human Monoclonal Antibody); and U.S. Pat. No. 4,946,778 (The Technique for Producing a Single Chain Antibody). Additionally, included are the following references for the antibodies: Biocca, S. et al., EMBO J., 9:101-108, 1990; Bird, R. E. et al., Science, 242: pp.423-426, 1988; Boss, M. A. et al., Nucl. Acids Res., 12: pp.3791-3806, 1984; Bukovsky, J. et al., Hybridoma, 6: pp.219-228, 1987; Daino, M. et al., Anal. Biochem., 166: pp.223-229, 1987; Huston, J. S. et al., Proc. Natl. Acad. Sci. USA, 85: pp.5879-5883, 1988; Jones, P. T. et al., Nature, 321: pp.522-525, 1986; Langone, J. J. et al., ed., “Methods in Enzymology”, Vol. 121 (immunological Techniques, Part I: Hybridoma Technology and Monoclonal Antibodies), Academic Press, New York, 1986; Morrison, S. et al., Proc. Natl. Acad. Sci. USA, 81: pp.6851-6855, 1984; Oi, V. T. et al., Biotechniques, 4:214-221, 1986; Riechmann, L. et al., Nature, 332: pp.323-327, 1988; Tramontano, A. et al., Proc. Natl. Acad. Sci. USA, 83: pp.6736-6740, 1986; Wood, C. et al., Nature, 314: pp.446-449, 1985; Nature, 314:452-454, 1985, or documents quoted therein (the disclosures of which are incorporated herein by reference).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they have the desirable biological activity (U.S. Pat. No. 4,816,567; Morrison et al., Proc. Natl. Acad. Sci. USA, 81: pp.6851-6855, 1984).

The monoclonal antibodies of the present invention can include those produced by hybridomas derived from mammalian animals and those produced by host cells transformed with the expression vector containing the antibody gene by the gene engineering methods.

The hybridoma producing the monoclonal antibody producing the anti-menin antibody can be made using myeloma cells and utilizing the cell fusion technique as follows:

That is, the hybridoma can be made by using menin or a fragment thereof as the sensitized antigen, immunizing with this according to the standard method, fusing the resultant immunized cells with parent cells known in the art by the standard cell fusion technique, and screening for cells producing the monoclonal antibody by the standard screening method. The preparation methods of menin or the fragment thereof and the immunization methods for mammalian animals can be carried out according to the techniques for preparing antisera containing polyclonal antibodies mentioned above. In this case, after immunization of the mammalian animal, an elevated level of the desired antibody is confirmed in the serum, and then the immunized cells are collected and subjected to cell fusion, and the preferred immunized cells include splenic cells particularly.

Myeloma cells of mammalian animals are used on the other hand as parent cells fused with the aforementioned immunized cells. Various cell lines known in the art can be used as these myeloma cells. Cell fusion of the immunized cells with the myeloma cells can be carried out basically according to the methods known in the art, for example, Kohler and Milstein's method (Kohler G and Milstein C, Methods Enzymol., 73: pp.3-46, 1981).

Described herein below is the production of antibodies, including embodiments of monoclonal antibodies. It goes without saying that the monoclonal antibody to be used in the present invention may be a monoclonal antibody obtained by utilizing cell fusion techniques with myeloma cells. The monoclonal antibodies can be prepared, for example, according to the following steps:

(1) Preparation of immunogenic antigens (immunogens),

(2) Immunization of animals with immunogenic antigens,

(3) Preparation of myeloma cells,

(4) Cell fusion between antibody-producing cells and myeloma cells,

(5) Selection and cloning of hybridomas (hybrid cells), and

(6) Production of monoclonal antibodies.

(1) Preparation of immunogenic antigens can be performed as follows:

The antigen as used herein includes isolated wild type menin polypeptides or fragment derivatives thereof (which may include partial domain polypeptides, fragments, partial peptides and synthetic polypeptides). Appropriate oligopeptides can be chemically synthesized based on information on the determined amino acid sequence of menin, and used as the antigen. Representative oligonucleotides include peptides having at least 5 consecutive amino acids among the amino acid residues residing in the region selected from the amino acid sequence composing menin or the partial fragment thereof.

The antigen may be used to immunize animals after being mixed with a suitable adjuvant without any modifications, but can be used after formation of immunogenic conjugates. For instance, the antigen used as an immunogen may be a fragment derived from menin, or a synthetic polypeptide fragment obtained via selecting a characteristic sequence region based on the amino acid sequence of menin, followed by design and chemical synthesis. The fragments may be coupled with various carrier proteins via suitable coupling agents to form immunogenic conjugates such as hapten-proteins. The immunogenic conjugates can be used to design monoclonal antibodies that can react with (or recognize) specific sequences exclusively. A cysteine residue or the like can be added to the polypeptide thus designed so as to readily prepare an immunogenic conjugate. To couple with a carrier protein or the like, the carrier protein is first activated. This activation may include incorporation of an activated binding group thereinto, etc.

The activated binding groups include (1) activated ester or activated carboxyl groups such as a nitrophenyl ester group, a pentafluorophenyl ester group, a 1-benzotriazol ester group, and an N-succinimido ester group, and (2) activated dithio groups such as a 2-pyridyldithio group and the like. The carrier proteins include keyhole limpet hemocyanin (KLH), bovine serum albumin (BSA), ovalbumin, globulin, polypeptides such as polylysine, bacterial components such as BCG and the like.

(2) Immunization of animals with the immunogenic antigen can be carried out as follows:

Animals may be immunized according to the methods known to those skilled in the art, and can be carried out according to techniques as described in, for example, Jikken Seibutsugaku Koza 14, Men-eki Seibutsugaku, Muramatu S. et al., ed., by Japanese Biochemical Society, Maruzen K.K., 1985; Zoku-Seikagaku Jikken Koza 5, Men-eki Seikagaku Kenkyuhou, ed., by Japanese Biochemical Society, Tokyo Kagaku-dojin Publishing Co., Inc., 1986; Shin-Seikagaku Jikken Koza 12, Bunshi Men-ekigaku III, Kougen, Koutai, Hotai, Tokyo Kagaku-dojin Publishing Co., Inc., 1992, and the like. Immunization can be performed in a mammal, for example, by one or more injections of an immunizing agent (and, if desired, an adjuvant). Typically, the immunizing agent and/or adjuvant will be injected in the mammal by multiple subcutaneous or intraperitoneal injections. The immunizing agent may include the aforementioned antigen peptides or related peptide fragments thereof. It may be useful to conjugate the immunizing agent to a protein known to be immunogenic in the mammal being immunized. Examples of such immunogenic proteins which may be employed include the aforementioned carrier proteins. The adjuvant to be used with the antigen includes, for example, Freund's complete adjuvant, Ribi adjuvant, pertussis vaccine, BCG, lipid A, liposome, aluminium hydroxide, silica, and the like. The immunization is carried out with suitable animals, including mice such as BALB/c, hamsters, and others. The antigen dose is, for example, approximately 1 to 400 μg/animal for mice. Generally, the antigen is injected intraperitoneally or subcutaneously into a host animal, followed by additional immunization by repeated courses wherein intraperitoneal, subcutaneous or intravenous administrations are carried out approximately 2 to 10 times at 1- to 4-week intervals, preferably 1- to 2-week intervals. For immunization, BALB/c mice, as well as F1 mice between BALB/c mice and other strain mice, etc. can be used. As required, the levels of animal immunization can be assessed by constructing an antibody titer measuring system followed by measuring the titer of an antibody. The antibody of the present invention may include those obtainable from such immunized animals, for example, anti-sera, polyclonal antibodies, etc.

(3) The preparation of myeloma cells can be carried out as follows:

Immortal cells (tumor cell lines) to be used for cell fusion can be selected from non-immunoglobulin-producing cell lines. The cell lines to be used for cell fusion may include, for example, P3-NS-1-Ag4-1 (NS-1, Eur. J. Immunol., 6:511-519,1976), SP-2/o-Ag14 (SP-2, Nature, 276:269-270, 1978), P3-X63-Ag8-U1 (P3U1, Curr. Topics Microbiol. Immunol., 81:1-7, 1978), P3-X63-Ag8 (X63, Nature, 256:495-497, 1975), P3-X63-Ag8-653 (653, J. Immunol., 123:1548-1550, 1979) which are derived from a murine myeloma MOPC-21 cell line, and the like. Murine myeloma cell lines resistant to 8-azaguanine can be sub-cultured in a medium for cell culture, such as Dulbecco's modified Eagle's medium (DMEM) or RPMI-1640 medium, supplemented with antibiotics such as penicillin, amikacin or the like, fatal calf serum (FCS) or the like and 8-azaguanine (for example, 5 to 45 μg/ml). The specified number of cell lines can be prepared by passage in the normal medium 2 to 5 days prior to cell fusion. The cell lines to be used may be cultured on a normal medium after the frozen and preserved cell lines have been completely thawed at approximately 37° C. and have been washed with a normal medium such as RPMI-1640 three or more times, and the specified number of cell lines may be prepared.

(4) The cell fusion between antibody-producing cells and myeloma cells can be carried out as follows:

After animals such as mice are immunized according to the above step (2), their spleens are taken out 2 to 5 days after final immunization, and the spleen cell suspension is obtained. In addition to the spleen cells, lymph node cells at various sites of the body can be obtained and used for cell fusion. More specifically, cell fusion is carried out, for example, in the standard nutrient culture medium in the presence of a cell fusion accelerator. As the culture media used for cell fusion, it is possible to use RPMI 1640 medium, MEM medium and others which are suitable for growth of the above myeloma cells as well as common culture media used for such cell cultures, and is also possible to admix serum fluid supplements such as fetal calf serum (FCS) and the like. Thus, the spleen cell suspension thus obtained and the myeloma cell lines obtained according to the above step (3) are placed in a cell culture medium such as a minimal essential medium (MEM), DMEM and RPMI-1640 medium followed by addition of a cell fusion accelerator, e.g., polyethylene glycol. Cell fusion accelerators widely known in the art can be used, including inactivated HVJ (Hemagglutinating virus of Japan, “Sendai virus”) and the like. Preferably, 0.5 to 2 ml of 30 to 60% polyethylene glycol can be added. Polyethylene glycol with a molecular weight of 1,000 to 8,000 can be used, more preferably, polyethylene glycol with a molecular weight of 1,000 to 4,000. The concentration of polyethylene glycol in the fusion medium is preferably, for example, 30 to 60%. As required, a small amount of dimethyl sulfoxide or the like is added to promote fusion efficiency. The ratio of spleen cells (lymphocytes): myeloma cell lines to be used for fusion is preferably 1:1 to 20:1, and preferably falls within 4:1 to 10:1.

In cell fusion, the target fusion cells (hybridomas) are formed by thoroughly mixing the given quantity of immunized cells and myeloma cells in the culture medium, and adding a PEG solution prewarmed at approx. 37° C. (e.g., an average molecular weight of approx. 1000 to 6000) typically at an concentration of 30 to 60% (w/v), followed by mixing. Subsequently, a cell fusion accelerator and the like which are not preferable for cell growth are removed by repeating the manipulation that an appropriate culture media is added sequentially followed by centrifugation to remove the supernatant.

The fusion reaction is carried out for 1 to 10 min, and subsequently a culture medium such as RPMI-1640 medium is added thereto. Fusion reaction can be done several times. After fusion reaction, cells are separated by centrifugation and transferred to the selection medium.

(5) Selection and cloning of hybridomas (hybrid cells) can be carried out as follows:

Selection media includes, for example, conventionally known “HAT medium”, i.e., FCS-containing MEM, RPMI-1640 medium, etc., supplemented with hypoxanthine, aminopterin, and thymidine. The culture in the above HAT medium is continued for a sufficient time period (typically several days to several weeks) until cells other than the target hybridomas (non-fused cells) die out. The replacement method for the selection medium can be generally carried out by replenishing an equivalent to the volume dispensed to the culture plate on the next day, and thereafter replacing the medium by half an amount every 1 to 3 days. The replacement can be modified depending on situations. Eight to sixteen days after fusion, the medium may be replaced every 1 to 4 days with conventionally known “HT medium” wherein aminopterin is excluded. As a feeder cell, for example, murine thymocyte can be used, which is sometimes effective.

The culture supernatant in culture wells with vigorously growing hybridomas is screened by using the given peptide fragment as the antigen or by using a labeled anti-mouse antibody for measuring target antibodies, with assay systems such as radioimmunoassay (RIA), enzyme-linked immunosorbent assay (ELISA), fluorescence immunoassay (FIA), luminescence immunoassay (LIA), Western blotting etc., or a fluorescence activated cell sorter (FACS), etc. The target antibody-producing hybridoma is cloned. Cloning is carried out by picking up colonies in agar media or by a limiting dilution. The limiting dilution is preferred. It is preferred that the cloning is performed multiple times. It is possible that the monoclonal antibody-producing hybridomas thus produced are subcultured in the standard culture medium. It is also possible to preserve the hybridomas in nitrogen liquid for a long time.

(6) The production of monoclonal antibodies can be carried out as follows:

In order to acquire the monoclonal antibody from the hybridoma, various techniques can be employed. Such techniques include the method where the hybridoma is cultured according to the standard method and a desired monoclonal antibody can be obtained from its culture supernatant, and the method where the hybridoma is inoculated into a histocompatible mammalian animal, propagated as an ascites tumor and the antibody can be isolated from its ascitic fluid. The former method is suitable for a highly purified antibody, and the latter is suitable for a large scale production of the antibody.

Thus, the obtained hybridoma lines can be cultured in an appropriate medium for growth such as MEM, RPMI-1640 medium containing FCS, and desired monoclonal antibodies can be obtained from the culture supernatants thereof. Large amounts of monoclonal antibodies can be produced by propagating hybridomas as ascites tumors, etc. In this case, each hybridoma is implanted intraperitoneally in an animal having the same histocompatibility as the strain from which the myeloma cells are derived for propagation. Or the hybridomas can be implanted in nude mice for propagation followed by collecting the monoclonal antibody produced in the ascites fluid of the animal. A mineral oil such as pristane (2,6,10, 14-tetramethylpentadecan) etc., can be administered in the peritoneal cavity in the animal prior to the implantation of the hybridomas, which are propagated after the treatment, and subsequently the ascites fluid can be collected. The ascites fluid can be used as the monoclonal antibody as it is, or by purifying by methods known in the art such as salting out the ammonium sulfate precipitation method, gel filtration methods by Sephadex etc., ion exchange chromatography, electrophoresis, dialysis, ultrafiltration, affinity chromatography methods, high performance liquid chromatography methods and the like. Preferably, the ascites containing the monoclonal antibody can be isolated and purified by ammonium sulfate fraction followed by an anion exchange gel such as DEAE-Sepharose and an affinity column such as a protein-A column. In particular, preferably included are the affinity chromatography immobilizing the antigen or the antigenic fragment (for example, synthetic peptides, recombinant antigenic proteins or the sites specifically recognized by the peptide or antibody), the affinity chromatography immobilizing protein-A, the hydroxyapatite chromatography and the like.

Also, transgenic mice or other organisms, for example, other mammalian animals can be used for expressing antibodies such as humanized antibodies against the immunogenic polypeptide products of the present invention.

The antibody can be made by the gene recombinant techniques by determining the sequence of the antibody obtained in a large amount in such ways or utilizing the nucleic acid sequence encoding the antibody obtained from the hybridoma line. The nucleic acids encoding the monoclonal antibody can be isolated and sequenced by commonly used techniques, for example, by using an oligonucleotide probe capable of specifically binding to the gene encoding the heavy or light chain of the murine antibody. The DNA once isolated can be introduced into an expression vector in the manner as described above and incorporated into the host cells such as CHO, COS cells and the like. The DNA can be modified, for example by substituting the sequence encoding the constant domain of the human heavy or light chain for the homogenous murine sequence (Morrison et al., Proc. Natl. Acad. Sci. USA, 81:6581, 1984). Thus, it is possible to prepare the chimera antibody or hybrid antibody having the desired binding specificity. Also, the antibody can be prepared into the chimera or hybrid antibody by applying the chemical protein synthesis techniques including the use of a condensation agent described below.

Humanized antibodies can be made by methods known in the art (for example, Jones et al., Nature, 321: pp.522-525, 1986; Riechmann et al., Nature, 332: pp.323-327, 1988; Verhoeyen et al., Science, 239: pp.1534-1536, 1988). Human monoclonal antibodies can also be made by methods known in the art, and human myeloma cells and human-mouse heterogenous myeloma cells for production of human monoclonal antibodies are also known in the art (Kozbor, J. Immunol., 133: p.3001, 1984; Brodeur et al., Monoclonal Antibody Production Techniques and Applications, pp.51-63, Marcel Dekker, Inc., New York, 1987. The methods for producing by-specific antibodies are also known in the art (Millstein et al., Nature, 305: pp.537-539, 1983; WO93/08829; Traunecker et al., EMBO, 10: pp.3655-3659, 1991; Suresh et al., “Methods in Enzymology”, Vol. 121, p.210, 1986).

Further, antibody fragments such as Fab, Fab′, and F(ab′)₂ obtained by treating these antibodies with enzymes such as trypsin, papain, pepsin and the like, and by reducing in some cases, may be used.

The antibodies can be used for known optional test methods such as competitive binding assay, direct and indirect sandwich assay and immunoprecipitation assay (Zola, Monoclonal antibodies: A Manual of Techniques pp.147-158, CRC Press, Inc., 1987).

To conjugate the antibody with detectable atomic group, it is possible to use the given methods known in the art, which include, for example, the methods described in David et al., Biochemistry 13: pp.1014-1021, 1974; Pain et al., J. Immunol. Meth., 40: pp.219-231, 1981; and “Methods in Enzymology” Vol. 184: pp.138-163, 1990). As the antibodies provided with labels, it is possible to use IgG fractions and a specific binding site, Fab′ obtained by digesting with pepsin followed by reducing. Examples of the labels in these cases are enzymes described below (peroxidase, alkali phosphatase or β-D-galactosidase etc.), chemical substances, fluorescent substances or radioisotopes.

Detection/measurement in the present invention can be carried out by immunostaining, e.g., tissue or cell staining, immunoelectron microscopy, immunoassay, e.g., competitive or non-competitive immunoassay, and radioimmunoassay (RIA), FIA, LIA, EIA, ELISA and the like can be used, B-F separation can be carried out, and the measurement can be carried out without performing the measurement. RIA, EIA, FIA and LIA are preferred, and sandwich type assay is included. For instance in the sandwich type assay, one side is the monoclonal antibody against the menin polypeptide of the invention and the other side is the polyclonal antibody against menin, and then either one is labeled so as to be detectable (of course, other combinations are possible, and a combination can be designed depending on purpose). The other antibody which can recognize the identical antigen is immobilized to the solid phase. A sample, the labeled antibody and the immobilized antibody are incubated to react sequentially, and the unbound antibody is removed followed by detecting the label. The amount of the detected label is proportional to the menin polypeptide antigen amount. Such assay is referred to as concurrent sandwich type assay, forward sandwich type assay or reverse sandwich type assay depending on the addition order of the insolublized antibody and labeled antibody. For example, washing, stirring, shaking, filtration or antigen pre-extraction and so on are appropriately employed in the measuring steps under the certain status. The other measuring conditions such as certain reagents, concentrations of buffers, temperature, or incubation time can be altered according to factors such as the antigen concentration in the specimen, the nature of the sample specimens and the like. Those skilled in the art can perform the measurement by appropriately selecting the effective optimal condition for each measurement while using the standard experimental methods.

Numerous carriers capable of immobilizing the antigen or antibody have been known and can be used in the present invention by appropriately selecting therefrom. As carriers, those used for the antigen antibody reaction are diversely known, and in the invention, it is of course possible to use those selected from those known in.the art. Those particularly suitably used include, for example, glasses e.g., activated glass such as aminoallylsilyl glass, porous glass, inorganic materials such as silica gel, silica-alumina, alumina, magnetizing iron, magnetizing alloy and the like, polyethylene, polypropylene, polyvinyl chloride, polyvinylidene fluoride, polyvinyl, polyvinyl acetate, polycarbonate, polymethacrylate, polystylene, stylene-butadiene copolymer, polyacrylamide, crosslinked polyacrylamide, stylene-methacrylate copolymer, polyglycidylmethacrylate, acrolein-ethyleneglycol dimethacrylate copolymer, etc., crosslinked albumin, collagen, gelatin, dextran, agarose, crosslinked agarose, natural or modified celluloses such as cellulose, microcrystalline cellulose, carboxymethylcellulose, cellulose acetate etc., crosslinked dextran, polyamides such as nylon, organic polymers such as polyurethane, polyepoxy resin etc., those by further emulsified polymerization thereof, those a functional group of which is introduced by a silane coupling agent in erythrocytes and cells if necessary, such as silicon gum, etc.

Furthermore, included are surfaces of solid substances (objects) such as filter paper, bead, tube, cuvette, inner walls of beakers, cells made up of synthetic materials such as test tube, titer plate, titer well, microplate, glass cell, synthetic resin cell, etc., glass bar, bar made up of synthetic materiel(s), bar thickened or thinned at the end, a bar attached round or flat prong at the end, thin plate bar, etc.

To these carriers, the antibodies can be bound, and preferably, it is possible to bind the anti-menin antibodies (including antisera and purified antibodies) and anti-menin monoclonal antibodies which react specifically for the antigens obtained in the present invention. The binding of the carriers to those involved in these antigen antibody reactions can be carried out by the physical techniques such as absorption, etc., or chemical methods using the condensation agent or using those activated, and by the techniques utilizing chemical binding reactions to each other. The labels include enzymes, enzymatic substrates, enzyme inhibitors, prosthetic groups, coenzymes, enzyme precursors, apoenzymes, fluorescent substances, dye substances, chemical luminescence compounds, luminescent substances, chromophoric groups, magnetic substances, metallic particles such as gold colloid etc., non-metallic element particles e.g., selen colloid, radioactive substances, and the like. The enzymes include dehydrogenase, redox enzymes such as reductase and oxidase, transferases which catalyze transferring, for example, amino, carboxyl, methyl, acyl and phosphate groups, hydrolytic enzymes which hydrolyze, for example, ester, glycoside, ether, peptide bonds, etc., lyase, isomerase, ligase and the like. Multiple enzymes can be compositively utilized for the detection. For instance, enzymatic cycling can also be utilized. Representative isotopes for labeling radioactive substances includes [³²P], [¹²⁵I], [¹³¹I], [³H], [¹⁴C], [³⁵S] and the like. Representative enzyme labeling includes peroxidase such as horseradish peroxidase, galactosidase such as Escherichia coli β-D-galactosidase, maleate dehydrogenase, glucose-6-phosphate dehydrogenase, glucose oxidase, glucoamylase, acetylcholine esterase, catalase, alkali phosphatase such as bovine small intestine alkali phosphatase and Escherichia coli alkali phosphatase, and the like. In the cases of using alkali phosphatase, measurement can be performed based on the fluorescence or luminescence generated by utilizing substrates such as the umbelliferone derivative such as 4-methylumbelliferyl phosphate, phosphorylated phenol derivatives such as nitrophenyl phosphate, enzymatic cycling system utilizing NADP, luciferin derivatives, dioxetan derivatives and the like. Luciferin-luciferase system can also be utilized. In the cases of using catalase, since oxygen is generated by reacting with hydrogen peroxide, the oxygen can be detected with an electrode. The electrodes can be glass electrodes, ion electrodes using an insoluble salt membrane, liquid membrane type electrodes, polymer membrane electrodes and the like.

Enzyme labeling can be replaced with a biotin-labeled material and enzyme-labeled avidin (streptoavidin). Detection sensitivity enhancing methods known in the art can be appropriately employed, for example, using the biotin-avidin system using secondary antibodies such as an antibody against an anti-galectin antibody. Multiple different labels can be used. In such cases, multiple measurements can be carried out continuously or discontinuously, and simultaneously or separately.

In the present invention, combinations of the enzyme reagents such as combinations of peroxidase such as horseradish peroxidase with 4-hydroxyphenyl acetate, o-phenylenediamine (OPD), tetramethylbenzidine (TMB), 5-aminosalicylate, 3,3-diaminobenzidinetetrahydrochloride (DAB), 3-amino-9-ethylcarbazole (AEC), tyramine, luminol, lucigenin luciferin and derivatives thereof, Pholad luciferin; alkali phosphatase with lumigen PPD, (4-methyl) umbelliferyl-phosphate, p-nitrophenol-phosphate, phenol-phosphate, bromochloroindolylphosphate (BCIP), AMPAK™ (DAKO), AmpliQ™ (DAKO), etc.; β-D-galactosidase, glucose-6-phosphate dehydrogenase with umbelliferyl galactoside such as 4-methylumbelliferyl-β-D-galactoside, nitrophenyl galactoside such as o-nitrophenol-β-D-galactoside; and glucose oxidase with ABTS etc., can be utilized for the formation of signals. It is possible to use those capable of forming quinol compounds such as hydroquinone, hydroxybenzoquinone, hydroxyanthraquinone, etc., lipo acids, thiol compounds such as glutathione etc., phenol derivatives, fetrocene derivatives by the actions of enzymes.

The fluorescence compounds or chemical luminescence compounds include fluorescein isothiocyanate (FITC), rhodamine derivatives such as rhodamine B isothiocyanate, tetramethylrhodamine isothiocyanate (RITC), tetramethylrhodamine isothiocyanate isomer R (TRITC), etc., 7-amino-4-coumarin-3-acetate, dansyl chloride, dansyl fluoride, fluorescamine, phycobiliprotein, acridinium salts, lumiferin, luciferase, luminol such as aequorin, imidazole, oxalate ester, rare earth chelate compounds, coumarin derivatives and the like. In order to detect the signals generated including luminescence and fluorescence, the detection can be performed visually, and can also be performed using an apparatus known in the art, and for example, a fluorometer, a plate reader and so on can be used. Also, in order to detect the signals from the radioisotopes, an apparatus known in the art can be used, and, for example, a gamma counter, scintillation and the like can also be used.

The labeling can be carried out by utilizing the reaction of a thiol group with a maleimide group, the reaction of pyridyldisulfide group with a thiol group, the reaction of an amino group with an aldehyde group, and the method can be applied by appropriately selecting from the methods known in the art and the methods which those skilled in the art can readily carry out, and the further modified methods thereof. It is possible to use the condensation agents capable of being used for manufacturing the above immunogenic complexes and the condensation agents capable of being used for binding to the carriers. The condensation agents include, for example, formaldehyde, glutaraldehyde, hexamethylene diisocyanate, hexamethylene diisothiocyanate, N,N′-polymethylenebisiodo-acetamide, N,N′-ethylenebismaleimide, ethylene glycol bissuccinimidyl succinate, bisdiazobenzidine, 1-ethyl-3-(3-dimethylaminopropyl) carbodiimide, succinimidyl 3-(2-pyridyldithio)propionate (SPDP), N-succinimidyl 4-(N-maleimidomethyl) cyclohexane-1-carboxylate (SMCC), N-sulfosuccinimidyl 4-(N-maleimidomethyl)cyclohexane-1-carboxylate, N-succinimidyl (4-iodeacetyl)aminobenzoate, N-succinimidyl 4-(1-maleimidophenyl)butyrate, N-(E-maleimidocaproyloxy)succinic imide (EMCS), iminothiolane, S-acetylmercaptosuccinic anhydride, methyl-3-(4′-dithiopyridyl)propionimidate, methyl-4-mercaptobutyrylimidate, methyl-3-mercaptopropionimidate, N-succinimidyl-S-acetylmercaptoacetate and the like.

According to the measuring methods of the present invention, it is possible to react the substance to be measured with the labeled antibody reagent such as the antiserum, purified antibody or monoclonal antibody labeled with the enzyme etc., and the antibody bound to the carrier sequentially or simultaneously. The order to add the reagents varies depending on the carrier system selected. When using beads of sensitized plastic, the measurement can be carried out by first adding the labeled antibody reagent such as the antiserum, purified antibody or monoclonal antibody labeled together with the sample specimen containing the substance to be measured into an appropriate test tube, and subsequently adding the beads of the sensitized plastic thereto.

In the measuring method of the present invention, the immunoassay is employed, wherein a solid phase carrier can be used by optionally selecting from various materials and forms such as balls, microplates, sticks, microparticles or test tubes, etc., made up of polystyrene, polycarbonate, polypropylene, or polyvinyl, which effectively adsorb proteins such as antibodies.

The measurement can be carried out in an appropriate buffer so as to maintain an optimal pH, for example approx. pH 4 to 9. Particularly proper buffers include, for example, acetate, citrate, phosphate, Tris, triethanol amine, borate, glycine, carbonate, Tris-hydrochloride and veronal buffer agents, and the like. The buffer agents can be used by mixing at a given rate to each other. It is preferred that the antigen antibody reaction is carried out at a temperature of approx. 0 to 60° C.

The incubation treatment of the labeled antibody reagent such as the antiserum, purified antibody or monoclonal antibody labeled with the enzyme and the antibody reagent bound to the carrier, and the substance to be measured can be carried out until reaching equilibrium, but the reaction can be stopped by separating the solid phase from the liquid phase after the limited incubation treatment at an extremely earlier time point than the time reaching equilibrium. Then, the degree of the existing label such as the enzyme can be measured in either the liquid or solid phase. The measuring manipulation can be carried out using an automated measuring apparatus, and the displayed signal generated by converting the substrate with the action of the enzyme can also be detected and measured using a luminescence detector, a photo detector, and the like. In the antigen antibody reaction, proper means can be performed such that the reagents used, the substance to be measured, and labels such as enzymes are stabilized and the antigen antibody reaction per se is stabilized. Moreover, in order to eliminate non-specific reactions, reduce inhibitory effects or activate the measuring reaction, it is also possible to add proteins, stabilizers, surfactants as described below, chelators, etc., into the incubation solution. As a chelator, more preferable is ethylenediamine tetra acetate (EDTA). Blocking treatments for preventing non-specific binding reactions which are usually employed in the art or are known to those skilled in the art may be carried out, and the reactions can be treated with, for example, normal sera or serum proteins of mammalian animals, albumin, hemoglobin, ovoalubmin (OVA), skim milk, milk fermented materials, collagen, gelatin, and the like. Such methods can be used without particular limitation so long as their purpose is to prevent non-specific binding reactions. Moreover, for washing the samples and the solid phases, the liquid can be used by appropriately selecting from the above buffer systems or brine, furthermore, it is possible to use by adding thereto those selected from the group consisting of cationic surfactants, anionic surfactants in addition to non-ionic surfactants such as Tween 20 (brand name), Tween 80 (brand name), NP-40 (brand name), Triton X100 (brand name), Briji (brand name), etc., amphoteric surfactants such as CHAPS.

As samples measured by the measuring methods of the present invention, it is possible to use all forms of solutions, colloid solutions, non-fluid samples and so on, and preferably they include samples derived from organisms, for example, all organs and tissues such as thymus, mammary tissue, ovary, uterus, prostate, colon, rectum, stomach, lung, bronchia, pancreas, liver etc., and malignant tumors of these organs and tissues, leukemia cells, blood, serum, plasma, joint fluid, saliva, amniotic fluid, urine, the other body fluids, cell culture liquids, tissue culture liquids, tissue homogenates, biopsy samples, tissues and cells and the like.

Setting particular conditions, manipulations and so on is not required for applying various analytical and quantitative methods including respective immunological measuring methods for the measuring methods of the present invention. It is enough to construct measuring systems relating to the subject substances and the substances having the activity substantially equivalent thereto by adding the standard technical consideration of those skilled in the art to the standard condition and manipulation method in each method.

Using the anti-menin antibody, particularly the monoclonal antibody, it is possible to perform epitope mapping, and it is possible to perform detection/measurement of respective menin and related peptide fragments thereof if using the antibody which recognizes respective epitope.

As mentioned above, the reagent containing the anti-menin antibody obtained as the polyclonal (including the antiserum) or monoclonal antibody can immunohistologically analyze the menin quantity using cancer cells extracted from the cancerated tissue as the specimen. That is, analysis of the menin quantity in subject cells enables the efficacy of the drug such as tamoxifen in the cells to be predicted.

The reagents for predicting the efficacy of drugs or agents for adjuvant hormonal therapy, used for the purpose of antineoplastically treating breast cancer, etc., and/or determining the efficacy of said drugs, are those containing an effective amount of the anti-menin antibody or containing anti-menin Ab as an effective component. The term “containing an effective amount of” means that anti-menin Ab is contained to an extent of concentration at which the expressed menin is detectable. The anti-menin antibody may include species capable of recognizing any menin protein regions which are not limited to epitopes.

For terms (words) and/or abbreviations used in the specification and in the drawings, they must conform with an “IUPAC-IUB Commission on Biochemical Nomenclature”, or are based on meanings of the terms which are commonly used in the art.

### EXAMPLES

Described below are examples of the present invention which are provided only for illustrative purposes and reference to specific embodiments thereof. Although these illustrative examples are provided for disclosing particular embodiments of the present invention, they should not be construed as limiting or restricting the scope of the present invention disclosed herein. It should be understood that various modes will be practicable based on the spirit of the present invention.

All the examples were or can be practiced using standard techniques well or conventionally known to those of ordinary skill in the art unless otherwise specified.

In the following examples, unless particularly indicated, specific operations and conditions for treatment were according to:

Sambrook, J., Fritsch, E. F. & Maniatis, T., “Molecular Cloning: A Laboratory Manual (2nd edition)”, Cold Spring Harbor Laboratory Press, Cold Spring Harbor, N.Y., 1989; Glover, D. M. et al. ed., “DNA Cloning”, 2nd ed., Vol. 1 to 4 (The Practical Approach Series), IRL Press, Oxford University Press, 1995 for DNA cloning, and Erlich H. A. (ed.), PCR Technology, Stockton Press, 1989; Glover D. M. et al. (ed.), “DNA Cloning”, 2nd ed., Vol. 1, (The Practical Approach Series), IRL Press, Oxford University Press (1995); Innis M. A. et al. (ed.), “PCR Protocols”, Academic Press, New York (1990) in the cases of using PCR methods. Furthermore, in instances where commercially available reagents or kits were used, protocols attached thereto or chemicals attached thereto are employed.

### Example 1

(RT-PCR Method)

Total RNA was extracted from breast cancer and normal mammary tissues, and cDNA was prepared. The following primers:

| | | |
|---|---|---|
| 5′-GAGCTGTCCCTCTATCCTCG-3′ | (sense): | SEQ ID NO:1 |
| 5′-TGACCTCAGCTGTCTGCTCC-3′ | (antisense): | SEQ ID NO:2 |

were used to amplify 286 base pairs by PCR, followed by detecting the expression of menin mRNA. β-Actin was used as an endogenous control. The PCR was carried out under the condition of 30 cycles wherein each cycle includes at 94° C. for 80 seconds, 55° C. for 80 seconds and 72° C. for 80 seconds, followed by at 72° C. for 15 min.

(Cell Culture)

MCF-7 cells (human breast cancer cell lines) and COS-7 cells were purchased from Human Science Research Source Bank. The cells were cultured in DMEM (ICN) medium to which 10% fetal calf serum (ICN) was supplemented.

(Plasmid Constructs)

Full-length menin cDNA was amplified by RT-PCR, and inserted into an expression vector, pcDNA3 (Invitrogen). The menin cDNA was identified by sequencing. The mutation (569delC) was introduced with a site-directed mutagenesis kit (Promega) to produce a variant menin. ERE-Luc (reporter gene: luciferase-labeled estrogen receptor-binding sequence), CMV-ERA (expression vector for estrogen receptor α-chain gene), and ER-GST (GST-labeled estrogen receptor) used were generously donated by Dr. Shin-ichi Hayashi, Saitama Cancer Center, Japan.

(Transcription Activity Analysis Using Transfected Cells) (Transient Transfection Assay)

The menin expression vector and the reporter gene (ERE-Luc) were co-transfected into MCF-7 cells according to the liposome method. Similarly, the menin expression vector, CMV-ERA and the reporter gene (ERE-Luc) were co-transfected. into COS-7 cells according to the liposome method. After culturing for 24 hours, the proteins were extracted from cells and assayed for their luciferase activity. The extracted proteins were measured for their protein concentration and β-galactosidase activity wherein β-galactosidase was co-transfected and the introduction efficiency was calibrated. Similar experiments were carried out with addition of 1 μM tamoxifen to the same system. Tamoxifen was purchased from Wako Pure Chemical Industries, Ltd.

(GST-Pull Down Assay)

The GST-ER fusion protein was produced in Escherichia coli, and partially purified with Glutathione Sepharose 4B beads (Amersham Pharmacia). On the other hand, the wild and variant type menin proteins were synthesized with ³⁵S-methionine labeling by the in vitro transcription/translation. Both proteins were reacted in vitro, and subjected to Glutathione Sepharose 4B beads. The protein adsorbed on the Glutathione Sepharose 4B beads was separated by SDS-PAGE, and compared to the sample reacted with GST alone.

(Western blotting)

The amino acid sequence (443-535) of menin was expressed as a GST-fusion protein in E. coli and used to immunize guinea pigs. Harvested IgG fractions were subjected to purification to give a menin-specific antibody. Proteins extracted from the cells were separated on 7.5% SDS-PAGE. After transferring onto PVDF membrane (Micropore), the above anti-menin antibody was reacted for one hour. Then, the second antibody, HRP-conjugated anti-GP IgG antibody (Pharmingen), was reacted for one hour, and subsequently the reaction was visualized by ECL method (Amersham Pharmacia).

(Immunohistological Staining)

Thin slices (4 μm) were prepared from breast cancer tissues embedded with paraffin. After removing paraffin, the microwave treatment was carried out (5 min×3) in citrate buffer. After cooling at room temperature, the endogenous peroxidase activity was inhibited using an aqueous 0.3% hydrogen peroxide solution. Next, blocking was performed with 6 drops of 5% bovine albumin (Sigma) for one hour, and then the anti-menin antibody (Santa Cruz) (first antibody capable of immunohistological staining) was reacted overnight (8 hours). After washing, the second antibody and 6 drops of Envision+ (Dako) wherein HRP was conjugated with polymers were reacted for one hour.

After 100 mg of a coloring agent, DAB (Dojindo), was thoroughly mixed in 150 ml of PBS, the slice was immersed in a solution containing 75 μl of 30% hydrogen peroxide (0.5 μl/ml) for 1 min, and immediately immersed into and washed with tap water. After the dehydration treatment, the slice was enclosed with a cover glass, and observed by a microscopy. The specimen where menin-positive cells with brown-colored nuclei were 50% or more of total cells was determined to be menin-positive.

(Results)

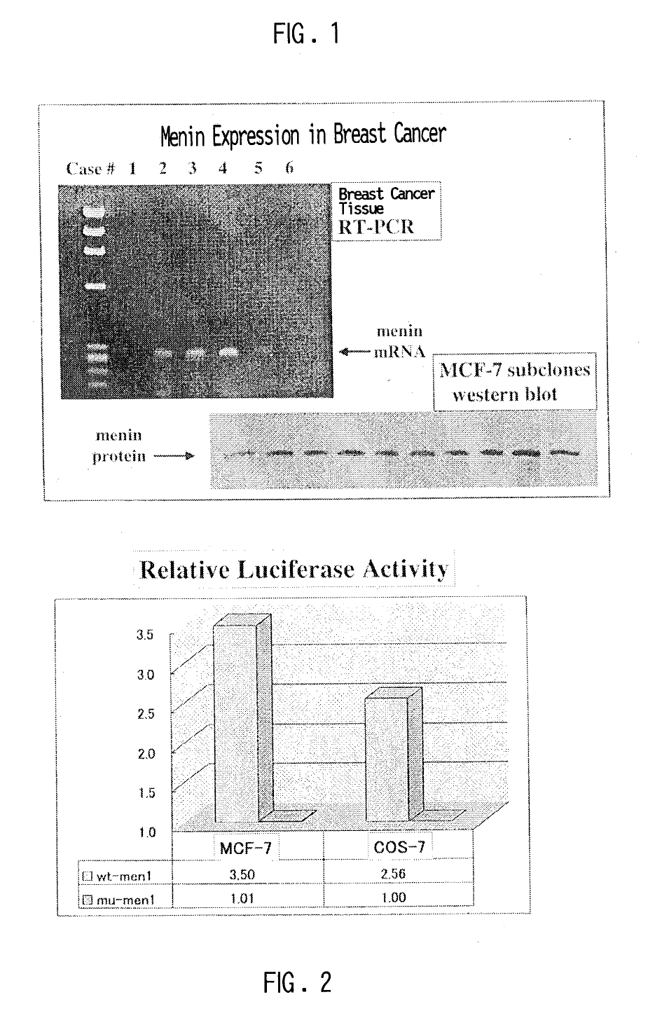

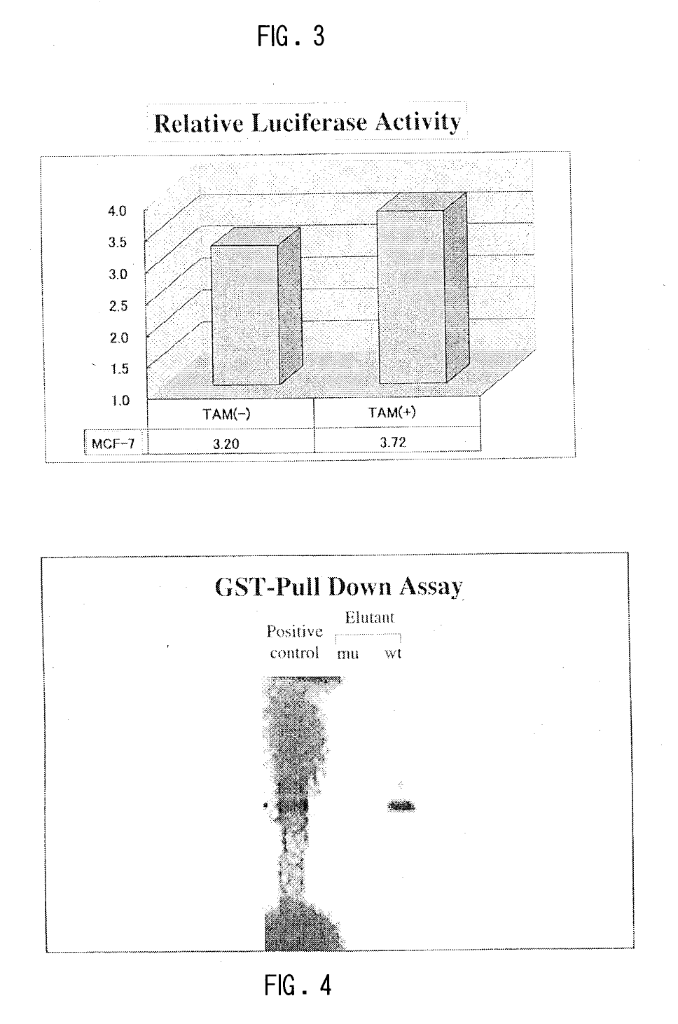

The expression of menin in breast cancer cells was analyzed by RT-PCR and Western blotting. As is apparently shown in FIG. 1, the expression of men1 mRNA was observed in breast cancer cells in the RT-PCR. The expression of menin in MCF-7 cells was detected in the Western blotting. Thus, the expression of menin was detected both at mRNA and protein levels. As a result of examining the regulation of menin for the estrogen receptor (ER)-transcription activity with the reporter gene assay using ERE-Luc, data exhibited 3 or more times stronger the luciferase activity of wt-men1-introduced cells than that of mu-men1-introduced cells (FIG. 2). As shown in FIG. 3, the luciferase activity was not inhibited by tamoxifen. In menin gene-transfected cells, the transcription-suppressive efficacy of the breast cancer therapeutic drug, tamoxifen, was not observed. Thus, it appears that menin is a tamoxifen resistant factor.

In GST-pull assay, menin was physically bound to the ER. As shown in FIG. 4, it was suggested that the wild type menin was directly bound to the ER to facilitate the transcription. The possibility was observed that menin was a transcription-coupling factor to facilitate the function of the ER. In contrast, the transcription-facilitating function was not observed for the mutant menin.

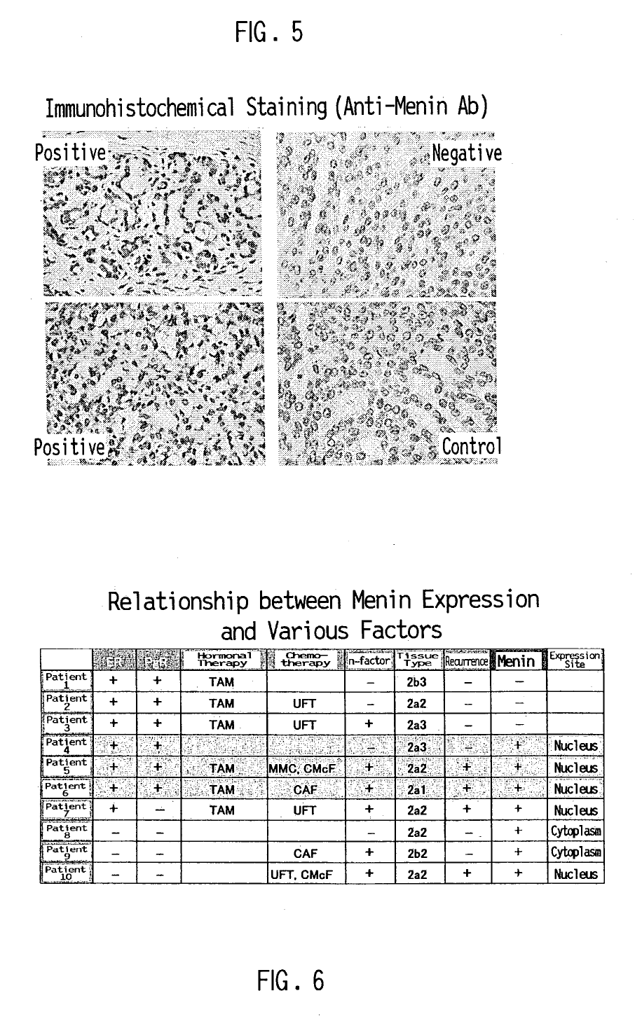

When the expression of menin in breast cancer tissues was examined with anti-menin antibodies (see FIG. 5), menin was negative in 3 of 10 patient cases, and no recurrence was observed by administrating tamoxifen.

On the other hand, in 5 cases where menin was positive, tamoxifen was administered in 3 cases, and cancer recurrence was observed in 4 cases. In the remaining 2 cases, menin was positive only in cytoplasm, and no cancer recurrence was observed (FIG. 6). Although metastasis to lymph node (n-factor) is also a predictor for cancer recurrence, Patient 3 has no recurrence even though n-factor is positive.

Thus, it has been suggested that menin is bound to the ER to facilitate the transcription activity and the analysis of menin expression enables the determination and/or assessment as to whether hormonal therapy is effective for malignant tumors such as breast cancer.

### Example 2

(Determination of Menin Binding Site by Two-Hybrid Assay)

For disclosing mechanisms by which menin enhanced the ER transcription activity to impair the efficacy of tamoxifen, it was assessed with the two-hybrid assay using a Gal4 system for an ER domain to which menin bound.

Each domain of the ER gene was incorporated into pCMX-Gal4 to form products: pCMX-Gal4-ER, pCMX-Gal4-ER-AF-1, pCMX-Gal4-ER-DBD, and pCMX-Gal4-ER-AF-2, respectively, wherein DBD is a DNA binding domain of the ER, AF1 is a region near to the DBD, and AF-2 is a ligand binding domain of the ER. The full-length menin gene was inserted into pCMX-VP16 to form pCMX-menin-VP16. Gal3x-TK/LUC vectors with an insert of a reporter gene, luciferase gene, were prepared.

COS cells were transfected at 50 to 70% using Lipofectamine (Life Technologies, Gaithersbueg, Md.) with Gal3x-TK/LUC vectors, pCMX-menin-VP16, and expression vectors for each ER domain. Twenty-four hours later, proteins were extracted from the transformed cells, and assayed for their luciferase activity to be defined as an index for molecular binding. The activity was calibrated using β-galactosidase. The experiments were conducted in triplicate. Data are shown as mean values with standard errors.

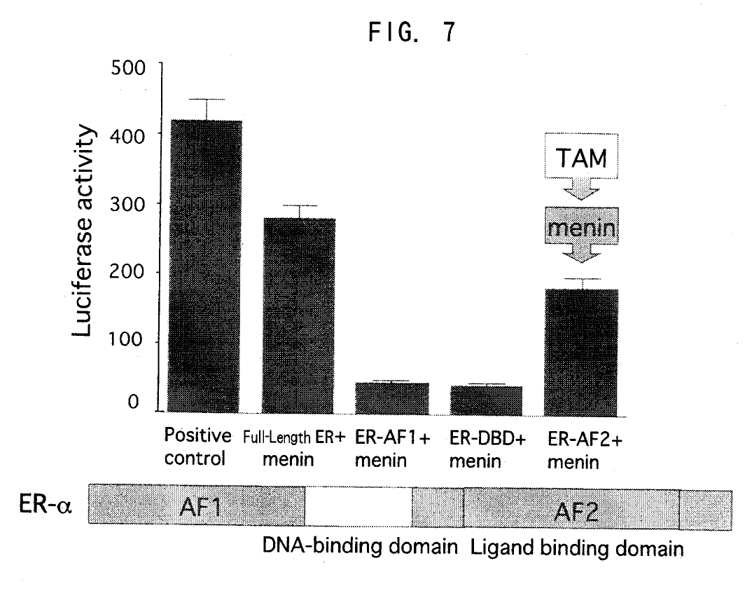

The results are shown in FIG. 7. In FIG. 7, \mathring{1} is co-transfected with Gal3x-TK/LUC+pCMX-Gal4-VP16 (positive control), \mathring{2} with Gal3x-TK/LUC+pCMX-Gal4-ER+pCMX-menin-VP16 (full-length ER+menin), \mathring{3} with Gal3x-TK/LUC+pCMX-Gal4-ER-AF-1+pCMX-menin-VP16 (AF-1 of the ER+menin), \mathring{4} with Gal3x-TK/LUC+pCMX-Gal4-ER-DBD+pCMX-menin-VP16 (DBD of the ER+menin), and \mathring{5}. With Gal3x-TK/LUC+pCMX-Gal4-ER-AF-2+pCMX-menin-VP16 (AF-2 of the ER+menin). As seen in FIG. 7, the menin-dependent ER transcription activity is non-enhanced when menin binds to the DNA binding domain of the ER while it is enhanced when menin binds to an estrogen- or tamoxifen-binding domain. It is apparent that menin enhances the ER transcription activity via binding to the AF-2 domain of the ER, thereby leading to the impairment of tamoxifen's efficacy.

### Advantageous Features of the Invention

It has also been elucidated that menin has an activity to facilitate the ER transcription activity and menin has a function as a resistant factor against tamoxifen which is used for the postoperative adjuvant hormonal therapy against malignant tumors such as breast cancer. Utilizing such findings, a manner to use menin as the marker for probing the efficacy of anti-tumor hormonal therapy has been developed. In the present invention, based on the presence or absence of menin (including the presence or absence of menin gene expression) the efficacy of estrogen antagonists such as tamoxifen in the hormonal therapy can be predicted and/or assessed. It can be considered possible that the efficacy of not only tamoxifen but also other drugs (toremifene, etc.) with similar biological activities can be predicted and/or assessed. Therefore, the presence or absence of menin expression (including menin gene expression) becomes an important decision-making factor or tool in selection of a drug used for breast cancer adjuvant hormone therapy.

While the present invention has been described specifically in detail with reference to certain embodiments and examples thereof, it would be apparent that it is possible to practice it in other forms. In light of the disclosure, it will be understood that various modifications and variations are within the spirit and scope of the appended claims.

What is claimed is:

1. Use of menin as a marker for probing or assessing the therapeutic efficacy of antineoplastic drugs on malignant tumors including breast cancer.

2. A method for the selection of a suitable therapeutic drug or for the prediction of therapeutic efficacy of said drug, which comprises employing menin as a marker for probing or assessing the therapeutic efficacy of said drug in order to select such a therapeutically-effective drug on adjuvant, or additional, hormone therapy after breast cancer surgery.

3. A reagent for probing or determining a therapeutically-effective drug on adjuvant, or additional, hormone therapy after breast cancer surgery, which comprises an element employing menin as a marker for probing or examining the therapeutic efficacy of said drug, said reagent being used for measuring or assaying menin in a subject sample to probe or determine the therapeutic validity of said drug on adjuvant, or additional, hormone therapy after breast cancer surgery.
